(19) United States
(12) Patent Application Publication
(10) Pub. No.: US 2023/0174913 A1
(43) Pub. Date: Jun. 8, 2023

(54) NOVEL HIGH-DENSITY MICROCARRIER RETENTION DEVICE FOR PERFUSION CULTURE AND METHOD OF USE THEREOF

(71) Applicant: GLOBAL LIFE SCIENCES SOLUTIONS USA LLC, Marlborough, MA (US)

(72) Inventors: Jianjun Yang, Shanghai (CN); Hongyan Yang, Shanghai (IN)

(21) Appl. No.: 17/998,461

(22) PCT Filed: Jul. 8, 2021
(86) PCT No.: PCT/EP2021/069061

§ 371 Date: Nov. 10, 2022

(30) Foreign Application Priority Data
Jul. 30, 2020 (CN) 202010750200.4

(51) Int. Cl. C12M 1/12; C12M 1/00

(52) U.S. Cl. CPC C12M 25/16; C12M 29/10

(57) ABSTRACT

The invention relates to the field of microcarrier perfusion culture of adherent cells. Specifically, the present invention relates to a high-density microcarrier retention device for perfusion culture of adherent cells, a microcarrier perfusion culture system for adherent cells containing the device, and methods of use thereof. The retention device of the present invention includes a sedimentation chamber, a pipeline connected to a bioreactor, a microcarrier retention filter membrane, a liquid backflushing device, an air backflushing device, a peristaltic pump and a pipeline connected to a receiver. The device has high efficiency in promoting the separation of microcarriers from cell culture medium and is helpful for perfusion culture of adherent cells and microcarriers. The retention device makes the culture volume in the bioreactor more flexible, can perform perfusion culture of 20%-100% of the maximum culture volume of the bioreactor, and the retention device can be linearly amplified according to the amplification of the bioreactor volume.

### TECHNICAL FIELD

The invention relates to the field of microcarrier perfusion culture of adherent cells. Specifically, the present invention relates to a high-density microcarrier retention device for perfusion culture of adherent cells, a microcarrier perfusion culture system for adherent cells containing the device, and methods of use thereof.

### BACKGROUND TECHNIQUE

The use of high-concentration microcarriers combined with perfusion culture technology to increase the cell density during inoculation, thereby increasing the virus titer of the upstream harvest liquid, is an important solution for the efficient production of vaccines. Current vaccine companies usually use adherent cells and microcarriers to perform perfusion culture in glass or stainless steel tank bioreactors for vaccine production. In order to effectively carry out perfusion culture, it is necessary to retain the microcarriers. Commonly used retention techniques include filtration, centrifugation, sedimentation, etc. In practice, these techniques have drawbacks, such as limited efficiency, high cost, pollution, etc.

U.S. Pat. US 5654197 provides a built-in gravity sedimentation device for cell perfusion culture. In this device, spent culture medium is pumped out through a sedimentation chamber located inside the bioreactor. The sedimentation chamber includes a hollow container, through a bottom opening of which the cells return to the stirred culture medium by gravity sedimentation, and through a top opening of which the spent culture medium without cells is pumped out of the bioreactor. The device requires that the medium fluid rate entering the sedimentation chamber through the bottom opening is significantly lower than the cell sedimentation rate.

Chinese patent application CN 102337200A provides a built-in microcarrier cell-medium separation device. In this device, the principle of gravity sedimentation is used to separate the microcarrier cells from the culture medium. The sedimentation chamber is designed with anti-disturbance partitions to form a low disturbance liquid environment, facilitating the sedimentation of microcarrier cells. The top of the sedimentation chamber is designed with a filter screen and a liquid backflushing device to prevent the microcarrier cells from leaving the sedimentation chamber.

Japanese patent application JPH06209761 provides a built-in microcarrier cell-medium separation device. In this device, the principle of gravity sedimentation is used to separate the microcarrier cells from the culture medium in a tubular sedimentation chamber. The tubular sedimentation chamber is designed to form a low disturbance liquid environment, facilitating the sedimentation of microcarrier cells.

Chinese patent application CN 107541464A provides a microcarrier cell-medium separation device. A layering funnel is provided in sedimentation columns. Two sedimentation columns are used in combination to keep the medium-microcarrier entrance of one sedimentation column higher than that of the other sedimentation column, and realize the function of controlling the liquid level by controlling the inlet flow rate and the outlet flow rate.

Prior art microcarrier cell-medium separation devices adopt a built-in way, such as those described above. The built-in separation device can ensure that the cells are in the growth conditions set in the bioreactor to the greatest extent. Although these separation devices can achieve the separation of microcarrier cells from culture medium to a certain extent, they suffer from significant limitations, including but not limited to: the inability to be scaled to large-volume bioreactors with tens or hundreds of liters, the inability to be applied to disposable bioreactors, the failure to be replaced inside the bioreactors due to clogging of the filter membrane, and the failure to use a larger perfusion rate to avoid the loss of microcarriers.

Therefore, there is still an urgent need to develop a new microcarrier cell-medium separation device in the field that does not suffer the above limitations. Such a separation device may be external to the bioreactor, facilitating replacement of the retention device, and/or designed as a disposable retention device. Such a device can accommodate a culture scale that can be amplified and can be connected to a disposable bioreactor to significantly improve the production efficiency of vaccines, viral vectors, oncolytic viruses, etc., and reduce production costs.

### BRIEF SUMMARY OF THE INVENTION

### High-Density Microcarrier Retention Device for Perfusion Culture

The invention provides a novel high-density microcarrier retention device for perfusion culture. Specifically, the high-density microcarrier retention device for perfusion culture is a high-density microcarrier retention device for adherent cell perfusion culture. This newly designed device has high efficiency in promoting the separation of microcarriers from cell culture medium, and is helpful for perfusion culture of adherent cells and microcarriers. This retention device makes the culture volume in the bioreactor more flexible, and can perform perfusion culture of 20%-100% of the maximum culture volume of the bioreactor.

The retention device of the present invention includes a sedimentation chamber, a pipeline connected to a bioreactor, a microcarrier retention filter membrane, a liquid backflushing device, an air backflushing device, a peristaltic pump and a pipeline connected to a receiver.

The sedimentation chamber is usually a cylinder or any shape with a smooth inner wall, and is made of various materials that meet the requirements of cell culture, such as plastic, metal, and glass. The bottom of the sedimentation chamber is connected to the bioreactor through a pipeline. The top of the sedimentation chamber is provided with a microcarrier retention filter membrane, the pore size of which is smaller than the diameter of the microcarrier, and is made from a suitable material such as stainless steel or polymer. The sedimentation chamber is connected to the liquid backflushing device and the air backflushing device respectively through a pipeline above the microcarrier retention filter membrane. The backflushing device is respectively composed of the corresponding backflushing pump or gas mass flow meter and connecting pipeline. Under the action of the peristaltic pump, the medium in the bioreactor enters the receiver through the sedimentation chamber. The receiver is used to receive the medium pumped from the retention device.

The microcarrier receives two forces in the sedimentation chamber, as shown in the figure below, the upward thrust (Fₘ) to the microcarrier generated by the fluid when the peristaltic pump pumps out the medium, and the downward gravity (Fₑ) of the microcarrier itself in the sedimentation chamber. In the perfusion culture, the flow rate of the peristaltic pump can be controlled to make the medium in the sedimentation chamber in the laminar flow zone. At this point Fₑ > Fₘ, the microcarrier will settle downward.

The sedimentation rate of the microcarrier satisfies the Stocks formula:

$$μ = \sqrt{\frac{4 ∗ {d}_{s} ∗ g ∗ \left({ρ}_{s} - ρ\right)}{3 ∗ ε ∗ ρ}}$$

where µ is the sedimentation rate of the microcarrier, dₛ is the diameter of the microcarrier, g is the acceleration of gravity, ρₛ is the density of the microcarrier, and ρ is the density of the medium. According to the Stokes formula, the sedimentation rate of the microcarrier can be calculated. Therefore, most of the microcarriers can be settled and returned to the bioreactor by adjusting the pumping rate of the medium and the height of the sedimentation chamber.

Therefore, when the sedimentation chamber is connected to the bioreactor through a pipeline, the upward pumping force of the peristaltic pump in the sedimentation chamber is smaller than the downward gravity of the microcarrier by controlling the pumping flow rate of the peristaltic pump. That is, the linear fluid rate of the medium in the sedimentation chamber is significantly smaller than the sedimentation rate of the microcarrier, so that the culture medium in the sedimentation chamber is in a laminar flow zone, causing the microcarrier to settle downward.

In one embodiment, the sedimentation chamber is connected to the bioreactor through one or more inclined or vertical pipelines. The angle α between the pipelines and the horizontal plane is about 60-90 degrees, for example, about 60, about 70, about 80, or about 90 degrees, preferably about 75 degrees.

When a small amount of microcarriers reaches the top of the sedimentation chamber, they can be retained by the filter membrane on the top of the sedimentation chamber to avoid the loss of microcarriers.

The microcarrier retention filter membrane is made of various materials that meet the requirements of cell culture, such as stainless steel, glass, or polymers. The inventors have found that by using vertical, inclined or curved retention walls, the microcarriers can be retained in the sedimentation chamber to a large extent without the clogging of the retention filter membrane. The term “retention wall” as used herein is defined as any barrier that the cell culture medium can pass through but the cell microcarriers cannot pass through and remains in the sedimentation chamber. Preferably, the retention wall contains one or more pores with a pore diameter smaller than the diameter of the microcarrier.

In one embodiment, the microcarrier retention filter membrane has a three-dimensional structure with one or more continuous or discontinuous vertical, inclined or curved retention walls. Such a three-dimensional structure may have an upper cross section and a lower cross section with the same or different shapes. For example, the shape of the upper cross section or the lower cross section may be a circle, an ellipse, a triangle, a square, a rectangle, a trapezoid, a pentagon, a hexagon, and any other regular or irregular polygons. In a further embodiment, the area of the upper cross section may be less than, equal to, or greater than that of the lower cross section. In a preferred embodiment, the area of the upper cross section is greater than or equal to that of the lower cross section. In another preferred embodiment, the lower cross section converges to a point. In one embodiment, a horizontal wall of the three-dimensional structure has a retention effect. In a particularly preferred embodiment, the horizontal wall of the three-dimensional structure has no retention effect. The microcarrier retention filter membrane without a horizontal retention wall can significantly prevent the clogging of the retention filter membrane. For example, the horizontal wall is made of polymer, glass or stainless steel, without one or more holes thereon, and does not allow any material (including cell culture medium) to pass through.

In a specific embodiment, the microcarrier retention filter membrane has an inverted cone structure, that is, a three-dimensional structure with an upper cross section larger than a lower cross section. The cone structure may be a circular cone, an elliptic cone, a triangular pyramid, a quadrangular pyramid, a pentagonal pyramid, and more pyramids. In one embodiment, the cone structure is an inverted pyramid three-dimensional structure.

In a further specific embodiment, the microcarrier retention filter membrane has an inverted cone parallel elongated three-dimensional structure, or a cylinder, cuboid or cube structure. For example, the microcarrier retention filter membrane has an inverted pyramid parallel extended three-dimensional structure.

In a further specific embodiment, the microcarrier retention filter membrane has a spherical or hemispherical three-dimensional structure.

The upper cross-section, lower cross-section and side view of the usable structure of the microcarrier retention filter membrane are shown in FIG. 6. These designs increase the retention area, and compared to the plane retention way, the vertical, inclined or curved retention wall can significantly reduce the attachment of microcarriers to the filter membrane.

In another embodiment, the liquid backflushing device is designed to backflush the medium in the pipeline above the microcarrier-retained filter membrane through a backflushing pump back to the sedimentation chamber. This can wash away a small amount of microcarriers adhering to the filter membrane and avoid the clogging of the filter membrane.

In another embodiment, the air backflushing device is designed to push all remaining culture medium and microcarriers in the retention device back to the bioreactor by means of sterile air through a gas mass flow meter. This prevents the cells from staying outside the bioreactor for a long time which causes the cell viability to decrease, and further avoids the clogging of the filter membrane.

The retention device of the present invention makes full use of the gravity sedimentation principle of the microcarrier and the retention principle of the filter membrane, which improves the separation efficiency of the microcarrier and the cell culture medium. Through the configuration of the retention device of the present invention, including the inclined or vertical pipelines connected to the bioreactor, the specific structure of the microcarrier retention filter membrane, the liquid backflushing device and the air backflushing device, there is little to no clogging of the filter membrane, and there is more flexibility and selectivity on the flow rate of medium entering the sedimentation chamber.

In one embodiment, the retention device of the present invention may be partially or wholly configured as a disposable device, for example made of plastic. The selection of a disposable device can be based on the following considerations: plug and play, shortened preparation time of the retention device; reduced unit operations, without equipment cleaning and sterilization; more convenient operation; simplified production process control; improved production efficiency; and saved production costs.

In another embodiment, the retention device of the present invention is a reusable device, for example made of stainless steel or glass.

### Microcarrier Retention Method

The present invention provides a method for retaining high-density microcarriers by using the retention device of the present invention. Specifically, the method includes the following steps:
i) pumping out culture medium and microcarriers from the bioreactor through the pipeline connected to the bioreactor to the retention device;
ii) harvesting the culture medium into the receiver through the pipeline connected to the receiver above the retention device and settling the microcarriers by gravity in the retention device;
iii) retaining a small amount of microcarriers still kept in the culture medium by the microcarrier retention filter member;
iv) backflushing the microcarrier retention filter membrane through the liquid backflushing device; and
v) pushing all remaining culture medium and microcarriers in the retention device back to the bioreactor by means of air through the air backflushing device.

In one embodiment, the method is performed by in an automated fashion, such as by a control program on a processor/controller. A user can freely set control program according to a daily perfusion rate, including the rate and time of pumping the medium and microcarriers into the retention device, the rate and time of harvesting the medium, the flow rate and time of liquid backflushing, and the flow rate and time of air backflushing.

The inventors uniquely conceived the use of air backflushing procedure in the method for retaining high-density microcarriers. By pre-setting various parameters of the air backflushing procedure, such as the flow rate and time of air backflushing, it is possible to prevent the cells from staying outside the bioreactor for a long time which causes the cell viability to decrease, ensuring that the cells are always in the optimal growth state. This further improves production efficiency.

In one embodiment, steps i)-v) are repeated one or more times.

In a preferred embodiment, the linear fluid rate of the culture medium in the sedimentation chamber of the retention device is less than the sedimentation rate of the microcarriers.

In the current field of gravity sedimentation of cell microcarriers, it is usually required that the linear fluid rate of the culture medium in the sedimentation chamber is significantly less than the sedimentation rate of the microcarriers, otherwise the retention filter membrane will be clogged. Through the unique configuration of the retention device according to the present invention, this necessary condition in the field is overcome, and there is no clogging of the filter membrane. Therefore, in another embodiment, the linear fluid rate of the medium in the sedimentation chamber of the retention device may be equal to or greater than the sedimentation rate of the microcarriers.

### Cell Microcarrier Perfusion Culture System

The present invention provides a cell microcarrier perfusion culture system, which includes the cell microcarrier retention device of the present invention, a bioreactor and a receiver.

In one embodiment, the cell microcarrier retention device of the present invention is externally connected to the bioreactor through a pipeline, so that the cell microcarrier retention device can be replaced at any time, depending on the length of the cell culture cycle and the clogging of the cell microcarrier retention filter. The external design is easy to disassemble and clean, and to replace the sterile cell microcarrier retention device to further extend the perfusion culture time and improve production efficiency.

The receiver is any container used to receive the culture medium recovered from the retention device.

In one embodiment, the cell microcarrier perfusion culture system may include one or more retention devices of the present invention, for example, 2, 3 or more retention devices. In one embodiment, multiple retention devices are connected to the bioreactor through separate or shared pipelines. In another embodiment, multiple retention devices are connected to the bioreactor through Y-joints or “all-in-one” tubing.

The size of the retention device can be scaled, and both small-volume and large-volume retention devices can be designed and produced.

The bioreactor can be from a few-liter scale small-volume bioreactor to a few hundred-liter scale large-volume bioreactor; it can also be a reusable glass or stainless-steel tank bioreactor, or a disposable bioreactor.

Currently, vaccine companies usually use adherent cells and microcarriers to culture in multiple small stainless steel bioreactors in parallel for vaccine production. Stainless steel tank bioreactors need to be cleaned and sterilized, which significantly reduces production efficiency. At the same time, multiple small bioreactors run in parallel, which also significantly increases labor intensity and pollution risks. Disposable bioreactors have been widely used in the production of antibody drugs, and have gradually entered the field of vaccine development and production in recent years. The use of disposable bioreactors for vaccine production has been a trend in the industry. Based on the need for efficient production of vaccines by perfusion culture, the design and development of microcarrier retention devices for perfusion culture suitable for disposable bioreactors, and the development of 50-200 L large-volume disposable bioreactors for vaccine efficient perfusion production have been very urgent. The retention device of the present invention can be used in combination with various specifications of disposable bioreactors, such as Cytiva XDR disposable bioreactors.

In one embodiment, the retention device can perform high-density perfusion culture of adherent cell microcarriers, wherein the concentration of the microcarriers ranges from 3 to 18 g/L.

### Cell Microcarrier Perfusion Culture Method

The present invention provides a cell microcarrier perfusion culture method by using the cell microcarrier perfusion culture system of the present invention. Specifically, the method includes the following steps:
i) placing cells and culture medium in a bioreactor for cultivation; and
ii) performing retention of the microcarriers by the microcarrier retention method of the present invention under the condition that fresh medium is supplemented by a feed pump, until sufficient cell culture products are obtained or the culture is completed.

In one embodiment, the cell microcarrier perfusion culture method is performed by an automated control program.

In one embodiment, the cell microcarrier retention device used in the cell microcarrier perfusion culture system adopts an external method, which can be easily replaced at any time depending on the length of the cell culture cycle and the clogging of the microcarrier retention filter membrane.

### DESCRIPTION OF THE DRAWINGS

According to the accompanying drawings, in combination with the specific embodiments of the present invention, the purpose, features and advantages of the present invention will become apparent. Those skilled in the art will understand that the dimensions of the components in the drawings are not drawn to scale, but are only for the purpose of explaining the present invention, and not limiting the scope of the present invention.

FIG. 1 is a schematic diagram of a configuration of the high-density microcarrier retention device (100) for perfusion culture of the present invention.

FIG. 2 is a schematic diagram of a cell microcarrier perfusion culture system including the high-density microcarrier retention device (100) for perfusion culture.

FIG. 3 shows an embodiment of the cell microcarrier perfusion culture system of the present invention.

FIG. 4 shows another embodiment of the cell microcarrier perfusion culture system of the present invention.

FIG. 5 shows the directions of movement of the microcarriers and cell culture medium in the pipeline connected to the bioreactor in the retention device of the present invention.

FIG. 6 shows various structures of the microcarrier retention filter membrane in the retention device of the present invention.

FIG. 7 shows the result of the bead to bead scale-up culture of Vero cells in 50 L and 200 L bioreactors.

FIG. 8 shows the result of perfusion culture of Vero cells in a 50 L bioreactor using the retention device of the present invention.

### DETAILED DESCRIPTION

The improved device and method of the present invention can be used in combination with any perfusion bioreactor or continuous cell culture system. Such a system design can maintain the entire culture process under optimal growth conditions to achieve high-density cell growth. These systems are particularly suitable for perfusion culture of adherent cells combined with microcarriers in a stirred bioreactor.

The terms “high density microcarrier retention device”, “cell microcarrier retention device”, “microcarrier retention device”, “retention device” or “device” are used interchangeably herein.

The terms “microcarrier-bound cell”, “cell microcarrier”, “microcarrier cell” or simply “cell” are used interchangeably herein and include any of cells, such as plant cells, insect cells and mammalian cells, which may be attached to the microcarrier and grow in a stirred suspension medium, and can settle by gravity in an unstirred medium with a reasonable sedimentation rate. More specifically, the cells to which the microcarrier is bound are adherent cells, usually mammalian cells, which are bound to the microcarrier particles. The microcarrier particles are, for example, glass, polystyrene, gelatin, dextran or cellulose beads, such as commercially available Cytodex-1 microcarriers, Cytodex-3 microcarriers or Cytopore microcarriers.

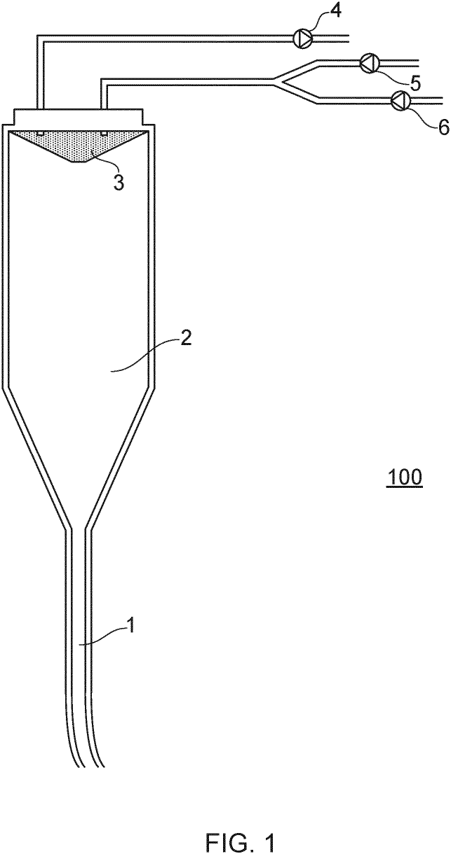

A high-density microcarrier retention device for perfusion culture of the present invention can be described according to FIG. 1. The device is an external independent sedimentation device for microcarrier culture, and other related devices are devices located in the bioreactor or devices externally and physically connected to the bioreactor. As an external device, the device (100) of the present invention is connected to the bioreactor through one or more inclined or vertical pipelines (1). The device (100) also includes a body in the form of a sedimentation chamber (2), which may be a cylinder or any shape with a smooth inner wall, and is made of various materials that meet the requirements of cell culture. A retention filter membrane (3) is installed on the interior top of the sedimentation chamber (2) to prevent the microcarriers from pumping out of the sedimentation chamber. On the top of the sedimentation chamber (2) and above the retention filter membrane (3), there are a plurality of pipelines respectively connected to a plurality of pumps. Under the action of a peristaltic pump (4), the cell microcarriers and the culture medium in the bioreactor enter the sedimentation chamber (2), and the culture medium leaves the sedimentation chamber through the retention filter membrane (3) and enters a receiver. A backflushing pump (5) has a liquid backflushing function, which performs liquid backflushing above the retention filter membrane (3) to prevent the retention filter membrane from clogging. A gas mass flow meter (6) and the pipeline connected thereto have an air backflushing function, which pushes all the culture medium and microcarriers in the sedimentation chamber back to the bioreactor by means of air through the pipeline (1).

The dvice shown in FIG. 1 can be used in conjunction with a processor to carry out an automated control program. The automated control program is a precise control program for cyclic periodic operation, including but not limited to the following subprograms:
1. Pump outing the culture medium and microcarriers from the bioreactor to the sedimentation device;
2. Harvesting the medium;
3. Settling the microcarriers in the device;
4. Retaining a small amount of microcarriers remaining in the culture medium by the microcarrier retention filter membrane;
5. Liquid backflushing the retention filter membrane; and
6. Pushing all the medium and microcarriers back into the bioreactor by means of air.

Optionally, the device of the invention includes a balance/loadcell for accurate and flexible culture volume automatic control.

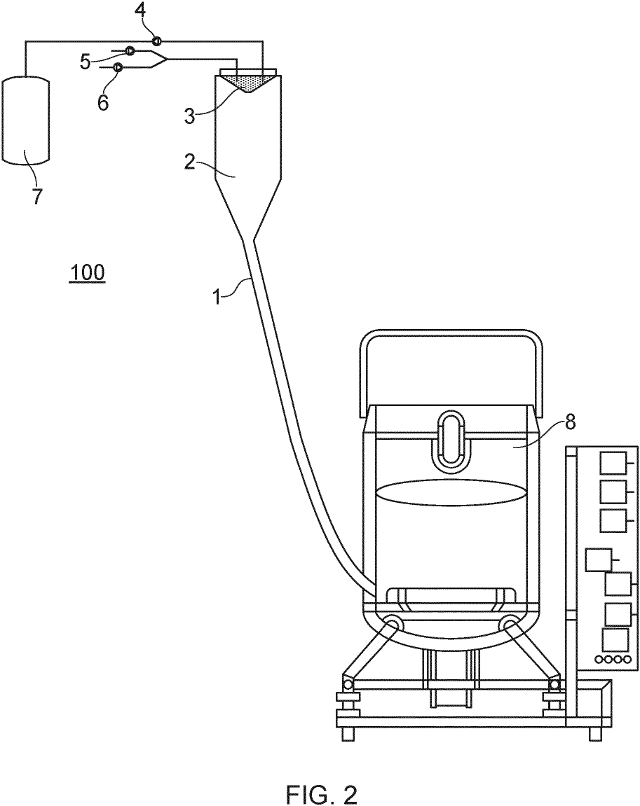

FIG. 2 shows an example of the cell microcarrier perfusion culture system of the present invention. The device (100) is fluidly connected to the bioreactor (8) through a pipeline (1). The bioreactor (8) can be a large-scale bioreactor. Alternatively, the bioreactor (8) is a disposable bioreactor. These bioreactors are well known to those skilled in the art and include many commercially available products such as Cytiva XDR disposable bioreactors.

Under the action of the peristaltic pump (4), the medium in the bioreactor enters the receiver (7) through the sedimentation chamber (2). After that, the medium in the receiver (7) can undergo the isolation and purification operations known in the art, such as centrifugation, filtration, chromatography etc., to obtain the target product.

In this example, the backflushing pump (5) operates regularly to perform liquid backflushing above the retention filter membrane (3) to prevent the retention filter membrane from clogging. In addition, the gas mass flow meter (6) operates regularly, and all the culture medium and microcarriers in the sedimentation chamber are pushed back to the bioreactor (8) through the pipeline (1) by means of sterile air.

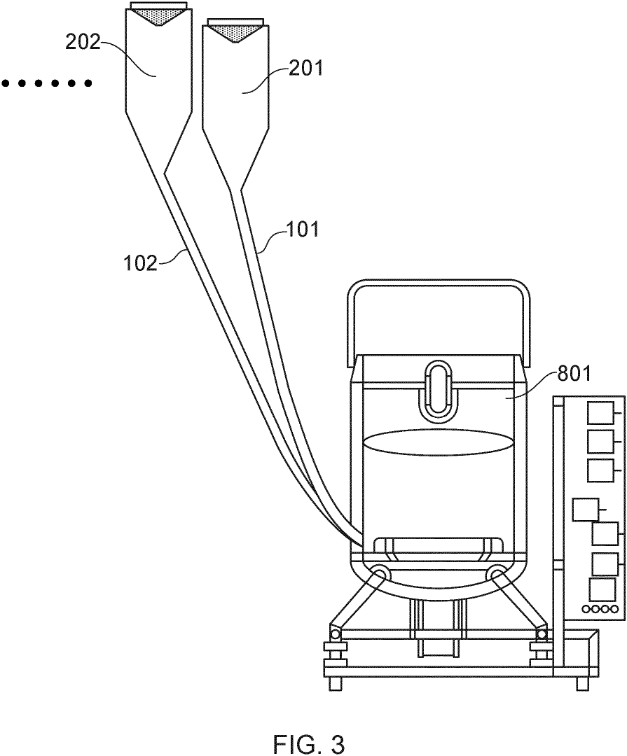

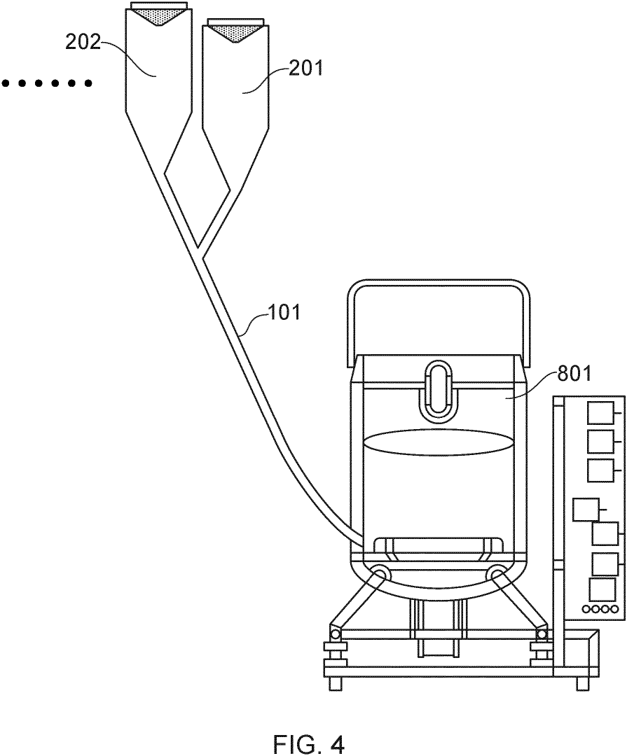

The retention device of the present invention is connected to the bioreactor and the receiver through pipelines, and such separation/connection is replaceable. That is to say, the retention device of the present invention can exist independently of the bioreactor and the receiver. In one embodiment, the cell microcarrier perfusion culture system may include one or more retention devices, for example, 2, 3 or more retention devices. FIGS. 3 and 4 show different ways of connecting the retention device to the bioreactor. In FIG. 3, a plurality of retention devices (201, 202, ...) are connected to the bioreactor (801) through separate pipelines (101, 102, ...). In FIG. 4, a plurality of retention devices (201, 202, ...) are connected to the bioreactor (801) through an “all-in-one” pipeline (101). Different connection ways provide flexibility for the configuration of the retention device of the present invention. Those skilled in the art can reasonably select the corresponding configuration according to the type of the bioreactor used, production efficiency, culture conditions, etc.

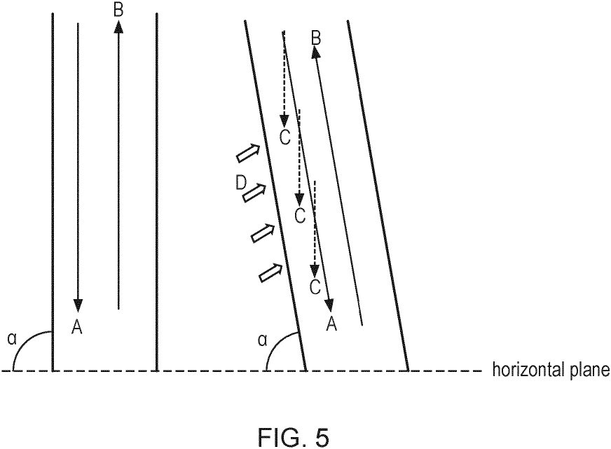

FIG. 5 shows the directions of movement of the microcarrier and cell culture medium in the pipeline connected to the bioreactor. The left panel shows that the sedimentation chamber is connected to the bioreactor through a vertical pipeline, and the angle α between the pipeline and the horizontal plane is 90 degrees. The right panel shows that the sedimentation chamber is connected to the bioreactor via an inclined pipeline (α<90 degrees). Specifically, as shown in FIG. 5, the cell microcarriers in the pipeline settle back to the bioreactor along the A direction, and the culture medium leaves the retention device along the B direction to be harvested. In the case of α<90 degrees, the cell microcarriers gather near the tube wall D along the direction of arrow C due to gravity, so that the cell microcarriers settle down along the tube wall D. In this case, it is easier for the cell microcarriers to settle in the pipeline connected to the bioreactor. Therefore, in a preferred embodiment, α<90 degrees. In either case, the retention of the filter membrane on the top of the sedimentation chamber can prevent the loss of microcarriers. This provides more options for the production process.

In the process of perfusion culture, the retention filter membrane has the risk of clogging. The retention device of the present invention adopts connecting pipelines with different angles in combination with an adjustable liquid flow rate, so that most of the microcarriers flow back to the bioreactor after a period of sedimentation time, which can significantly reduce the concentration of microcarriers in the sedimentation chamber and reduce the clogging of the retention filter membrane by microcarriers.

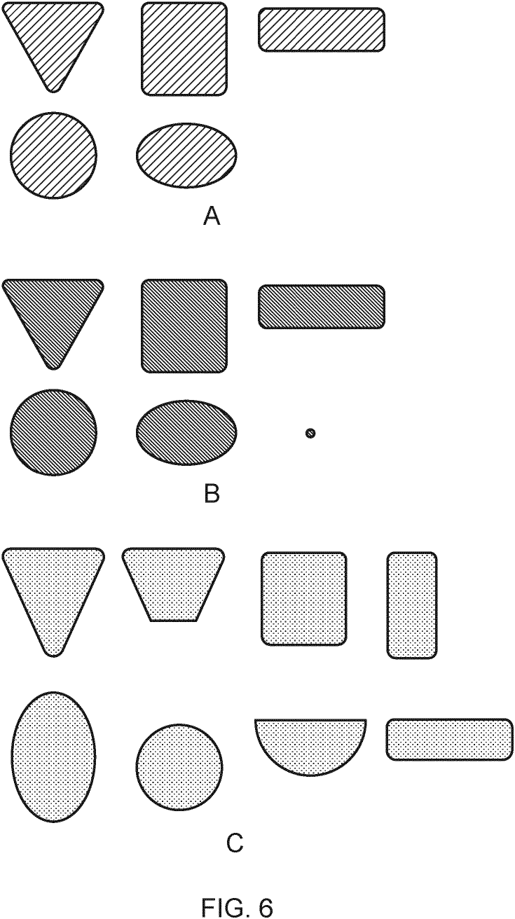

The retention filter membrane of the microcarrier retention device of the present invention adopts a unique three-dimensional structure to increase the retention area, improving the retention efficiency and preventing the clogging. The upper cross section (A), lower cross section (B) and side view (C) of the various three-dimensional structures of the retention filter membrane are shown in FIG. 6. The shapes A, B, and C can be combined in various suitable forms to form the three-dimensional structure of the retention filter membrane of the present invention. The retention filter membrane can have an inverted cone structure, that is, a three-dimensional structure with an upper cross section larger than a lower cross section. The cone structure may be a circular cone, an elliptic cone, a triangular pyramid, a quadrangular pyramid, a pentagonal pyramid, and more pyramids. In one embodiment, the cone structure is an inverted pyramid three-dimensional structure. In a further embodiment, the microcarrier retention filter membrane may have an inverted cone parallel elongated three-dimensional structure, or a cylinder, cuboid or cube structure. For example, the microcarrier retention filter membrane has an inverted pyramid parallel extended three-dimensional structure. In a further embodiment, the microcarrier retention filter membrane has a spherical or hemispherical three-dimensional structure. These designs increase the retention area, and compared to the plane retention way, the vertical, inclined or curved retention wall can significantly reduce the attachment of microcarriers to the filter membrane.

In addition, the liquid backflushing procedure can wash away a small amount of microcarriers adhering to the filter membrane to avoid the clogging of the filter membrane. The air backflushing procedure can push the microcarriers back into the bioreactor in a short time, avoiding the cell viability decline caused by the cells staying outside the bioreactor for a long time, and further avoiding the clogging of filter membrane. Through the use of device products and cell culture perfusion experiments, it is confirmed that the design of this microcarrier retention device has no clogging of filter membrane and has no effect on cell viability.

The device of the invention solves the problems of high-density microcarrier perfusion culture, and is especially suitable for large-scale bioreactors and disposable bioreactors. The device has been tested in the Cytiva Fast Trak laboratory and cooperative laboratories, using Vero cells and microcarriers to successfully carry out high-density microcarrier perfusion culture, and successfully scaled up the Vero cell microcarrier perfusion culture process to 50 L disposable bioreactors for producing rabies vaccine.

In a specific experiment, Vero cells and Cytodex-1 microcarriers were used to compare culture results of the batch culture mode of cell microcarriers in 50 L and 200 L XDR bioreactors with the perfusion culture mode of cell microcarriers in 50 L XDR bioreactors.

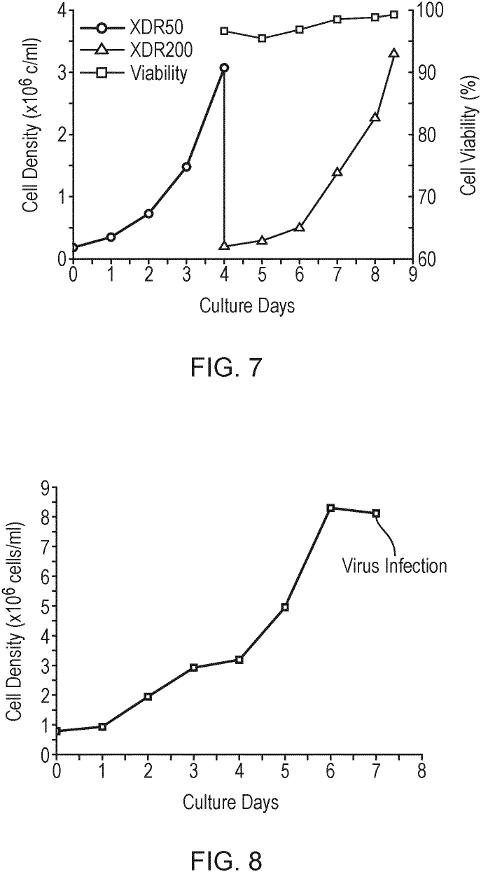

In the batch culture of cell microcarriers, lower microcarrier concentrations are usually used. For Vero cells, a Cytodex-1 microcarrier concentration of 2-3 g/L is usually used. Higher microcarrier concentrations require special control of environmental conditions or frequent medium changes. The inventors used 3 g/L Cytodex-1 microcarriers and the XDR50 bioreactor to culture Vero cells. By optimizing the culture conditions, the cell density can only reach 3 × 10⁶ cells/ml (FIG. 7, day 4). Through the microcarrier bead to bead scale-up culture, the Vero cell culture is scaled up to the XDR200 bioreactor, and the cell density can still only reach 3 × 10⁶ cells/ml (FIG. 7, days 8-9). The lower concentration of microcarriers and limited nutrient replenishment ways make the whole culture unable to obtain higher cell density, which in turn affects the virus titer in the harvested liquid after inoculation.

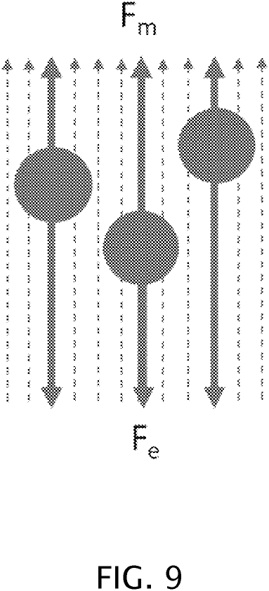

Through the novel microcarrier retention device for perfusion culture of the present invention combined with an XDR50 bioreactor, Cytodex-1 microcarriers are used for Vero cell perfusion culture. The microcarrier concentration can be increased to 12-18 g/L, making the cell density more than 8 × 10⁶ cells/ml (FIG. 8, day 6). The cell density is increased by almost 3 times through perfusion culture, which results in the efficient production of vaccines in disposable bioreactors.

Specifically, the cell microcarrier perfusion culture system of the present invention supported the perfusion culture of Vero cells with 12 g/L Cytodex-1 microcarriers in a 50L bioreactor for 22 days, so that the cell density exceeded 8×10⁶ cells/ml. The first 7 days was the growth period of cell perfusion culture, and the last 15 days was the collection period of rabies vaccine of perfusion culture. In the entire run, the retention rate of cell microcarriers was 100%.

1. A high-density microcarrier retention device for perfusion culture, the device comprising:
a sedimentation chamber;
a first pipeline connected to a bioreactor and the sedimentation chamber;
a microcarrier retention filter membrane located within the sedimentation chamber;
a pump and a second pipeline connected to a receiver and the sedimentation chamber; and
a liquid backflushing device and an air backflushing device, each connected to the sedimentation chamber.

2. The high-density microcarrier retention device for perfusion culture according to claim 1, wherein the high-density microcarrier retention device for perfusion culture is a high-density microcarrier retention device for adherent cell perfusion culture.

3. The high-density microcarrier retention device for perfusion culture according to claim 1 wherein the sedimentation chamber is connected to the bioreactor through one or more inclined or vertical pipelines, and the angle a between the pipelines and the horizontal plane is between 60-90 degrees.

4. The high-density microcarrier retention device for perfusion culture according to wherein the microcarrier retention filter membrane has a three-dimensional structure with one or more continuous or discontinuous vertical, inclined or curved retention walls.

5. The high-density microcarrier retention device for perfusion culture according to claim 4, wherein the three-dimensional structure has an upper cross section and a lower cross section with the same or different shapes.

6. The high-density microcarrier retention device for perfusion culture according to claim 5, wherein an area of the upper cross section is greater than or equal to that of the lower cross section.

7. The high-density microcarrier retention device for perfusion culture according to claim 5 wherein the lower cross section converges to a point.

8. The high-density microcarrier retention device for perfusion culture according to wherein a horizontal wall of the three-dimensional structure is configured to have a retention effect.

9. The high-density microcarrier retention device for perfusion culture according to claim 1, wherein the device is partially or wholly configured as a disposable device.

10. The high-density microcarrier retention device for perfusion culture according to claim 1, wherein the device is a reusable device.

11. The high-density microcarrier retention device for perfusion culture according to claim 1, wherein the microcarrier retention filter membrane is replaceable.

12. A method for retaining high-density microcarriers by using the high-density microcarrier retention device for perfusion culture according to claim 1 comprising the following steps:
i) pumping out culture medium and microcarriers from the bioreactor through the first pipeline connected to the bioreactor to the retention device;
ii) harvesting the culture medium into the receiver through the second pipeline connected to the receiver above the retention device and settling the microcarriers by gravity in the retention device;
iii) retaining a small amount of microcarriers still kept in the culture medium by the microcarrier retention filter member;
iv) backflushing the microcarrier retention filter membrane through the liquid backflushing device; and
v) pushing remaining culture medium and microcarriers in the retention device back to the bioreactor by means of air through the air backflushing device.

13. The method according to claim 12, wherein the method is performed by an automated control program.

14. The method according to claim 12, wherein steps i) - v) are repeated one or more times.

15. The method according to claim 12, wherein a linear fluid rate of the culture medium in the sedimentation chamber of the retention device is less than the sedimentation rate of the microcarriers.
